(19) United States
(12) Patent Application Publication
(10) Pub. No.: US 2011/0045068 A1
(43) Pub. Date: Feb. 24, 2011

(54) PHARMACEUTICAL FORMULATIONS FOR THE ORAL ADMINISTRATION OF PPI

(76) Inventors: Roberto Valducci, Savignano Sul Rubicone (IT); Tiziano Alighieri, Rimini (IT); Serozh Avanessian, Villa Verucchio (IT)

Correspondence Address: STETINA BRUNDA GARRED & BRUCKER, 75 ENTERPRISE, SUITE 250, ALISO VIEJO, CA, 92656, US

(21) Appl. No.: 12/734,491

(22) PCT Filed: Nov. 7, 2008
(86) PCT No.: PCT/EP2008/065127

§ 371 Date: May 5, 2010

(30) Foreign Application Priority Data
Nov. 9, 2007 (IT) FI2007A000253

(51) Int. Cl. A61K 9/28; A61K 31/4439; A61P 1/00

(52) U.S. Cl. 424/465; 424/474; 514/338

(57) ABSTRACT

The present invention relates to pharmaceutical formulations comprising a layer of compressed granules to facilitate the administration of oral PPI.

### FIELD OF THE INVENTION

The present invention applies to the field or pharmaceutical formulations for oral administration and particularly to formulations in which the active ingredients are proton pump inhibitors (PPD.

### STATE OF THE ART

It is common knowledge that proton pump inhibitors (PPI) are drugs containing active ingredients discovered relatively recently that have proved very effective in treating various gastroenterological diseases, thanks to their ability to reduce acid secretion by the stomach.

These drugs are normally well tolerated; they have a relatively short half-life in plasma but a persistent action in irreversibly inhibiting the enzyme H+,K(+)-ATPase that superintends the production of hydrochloric acid by the gastric parietal cells.

In the preparation of PPI formulations suitable for oral administration, however, a difficulty arises in the stabilisation of the active ingredient, which is done by adding alkaline substances and/or applying layers of “inert” coating to protect the active ingredient.

Moreover, since the active ingredient has to be absorbed at intestinal level, it is essential to add a gastroresistant layer to prevent the medicine from being inactivated by food or proteins in the stomach.

The need to achieve the above results makes it necessary to add potentially harmful products, as well as making the production process slower and implicating the use of solvents and/or “humid” processes in the preparation of the protective layer that are potentially hazardous and/or harmful to the environment.

Despite the very numerous studies conducted in the sector in an attempt to overcome the above-mentioned problems, none of the proposed solutions has so far proved entirely satisfactory and there is consequently an evident interest in the ability to provide pharmaceutical formulations capable of solving the aforesaid problem.

### SUMMARY OF THE INVENTION

This is a pharmaceutical formulation comprising an inner tablet containing the active ingredient (PPI) surrounded by a protective outer tablet and complete with a gastroresistant, or a protective and gastroresistant outer coating.

### DETAILED DESCRIPTION OF THE INVENTION

The present invention enables the above-described problem to be overcome thanks to pharmaceutical formulations comprising:
an inner tablet containing the active ingredient (PPI);
a protective outer tablet surrounding said inner tablet;
a gastroresistant, or protective and gastroresistant outer coating, and possibly also a protective coating inserted between the above-described inner tablet and protective outer tablet.

According to the invention, by active ingredient PPI we mean the known commercially-available compounds (such as omeprazole, pantoprazole sodium sesquihydrate, lansoprazole, rabeprazole sodium, esomeprazole magnesium dihydrate).

The inner tablet, comprising the active ingredient, is a conventional tablet obtained by compressing a preparation in granules, spraying a binder solution over a mixture of powders (including the active ingredient). These procedures can be implemented using various types of equipment, such as fluid beds or traditional wet granulators. In addition to the active ingredient, the mixture of powders also includes the usual surfactants, binders and disintegrants normally used for such purposes. Said additional ingredients may be chosen, for instance, from among the following: mannitol, povidone and its derivatives, calcium silicate, microcrystalline cellulose, sorbitol, lactose, starch and its derivatives.

The composition of the above-described tablet includes:

| | | | |
|---|---|---|---|
| active ingredient | 50-80% | preferably | 60-80% |
| surfactant | 1-15% | preferably | 3-10% |
| binders | 3-25% | preferably | 5-15% |
| disintegrants | 1-10% | preferably | 4-6% |
| diluent | 15-30% | preferably | 8-20% |

The outer protective tablet consists of granules produced using fluid bed technology or wet granulation with a conventional granulator, or direct compression.

The composition of said granules includes: polyalcohols, isomalt, microcrystalline cellulose, pharmaceutically-allowable soluble or insoluble polymers, and lubricants.

According to the invention, the polyalcohols might include: mannitol, polyisosorbate, xylitol and erythritol.

The pharmaceutically-allowable polymers might include, for instance: povidone, crospovidone, polyethylene glycol, polyvinyl alcohol, cellulose derivatives and modifications thereof, such as hydroxypropyl cellulose, hydroxyethyl cellulose, hydroxypropyl methyl cellulose, carragenin, carbopol, and so on.

The use of water-soluble polymers of the above-mentioned type enables the tablets to disintegrate rapidly, while using water-insoluble polymers makes the cores dissolve more slowly.

According to the invention, examples of the lubricants normally used for the purpose of preparing such pharmaceutical formulations include: magnesium stearate, talc, colloidal silica, and so on.

In this type of formulation, both the inner tablet (containing the active ingredient) and the outer (protective) tablet may also contain excipients such as: povidone, crospovidone, talc, lactose, mannitol, magnesium stearate, colloidal silica, etcetera.

The composition of the above-described protective outer tablet comprises:

| | | | |
|---|---|---|---|
| binders | 5-10% | preferably | 6-10% |
| disintegrants | 14-30% | preferably | 18-25% |
| diluents | 50-80% | preferably | 60-75% |
| lubricants | 1-5% | preferably | 1-3% |

Finally, the protective layer applied to the outer tablet consists of: pH-dependent films for protecting the final tablet against acid environments, such as methacrylic copolymer type A, B, C, hydroxymethyl cellulose phthalate, hydroxymethyl cellulose succinate, cellulose acetate trimellitate, polyvinyl acetate phthalate, cellulose acetate phthalate; or pH-independent films to facilitate its swallowing, for which cellulose derivatives are used (e.g. hydroxycellulose, hydroxyethyl cellulose and starch derivatives), dimethylaminoethyl methacrylate cationic polymers, methacrylic copolymer with aminoethyl methacrylate functional groups.

The formulations according to the invention are manufactured using known equipment and methods for the production of pharmaceutical formulations in tablet form and this, as mentioned above, is another advantage of the formulations according to the invention.

In particular, the granules containing the active ingredient are prepared according to the invention using a fluid bed or conventional granulator, then these granules are mixed with the necessary lubricant excipients, compressing the mixture into the form of a tablet (the inner tablet).

Likewise, the inert granules that constitute the protective outer tablet are prepared with a fluid bed or conventional granulator and the resulting granules, possibly mixed with other lubricant excipients, are compressed into the form of a tablet surrounding the previously-obtained inner tablet.

Another method that can be used is the direct compression of microcrystalline cellulose combined with isomalt and lubricated with magnesium stearate.

Finally, the completed tablet is covered with a film of gastroresistant coating, possibly preceded by a coating providing protection against humidity.

The above-described films are applied in drum mixers using an automated tablet-coating technology.

The formation of the protective layer obtained by dry compression enables the following advantages to be achieved:
it produces stable formulations without resorting to any addition of alkaline or antioxidant or buffer substances, or variable mixtures thereof, in order to stabilise the active ingredients;
it speeds up the production process, which can be implemented using well-established means for the manufacture of solid formulations for oral administration;
it restricts the need for solvents and/or wet processes in the preparation of the gastroresistant film, which are potentially hazardous to the process and/or harmful to the environment.

For the sake of completeness and a better understanding of the invention, several examples of formulations according to the invention and their related preparation are given below.

### Example 1

Rabeprazole is placed in a fluid bed with a top-spray insert and it is sprayed with a solution of povidone-polysorbate 80-ethanol (1;0.23;9).

After spraying with all the previously-described solution, the granules are dried inside said fluid bed.

After drying, the resulting granules are forced through a vibrating sieve fitted with a mesh with holes 1000 μm in diameter and mixed in a drum mixer with magnesium stearate as a lubricant; this mixture is then used to produce small tablets, 3 mm in diameter, with a unit composition, in mg, of:

| | | |
|---|---|---|
| Rabeprazole sodium | 10.00 | (86.96%) |
| Povidone | 0.81 | (7.04%) |
| Polysorbate 80 | 0.19 | (1.65%) |
| Magnesium stearate | 0.50 | (4.35%) |

Second inert granules are prepared separately in a fluid bed containing mannitol, which is sprayed with a solution of povidone-ethanol (1:9). After spraying with all of the previously-described solution, the granules are dried inside the same fluid bed.

After drying, the resulting granules are forced through a vibrating sieve fitted with a mesh with holes 1000 μm in diameter and mixed in a drum mixer with magnesium stearate as a lubricant.

Using a suitable compression machine, the previously-prepared inner tablets are covered with the second granules obtained as explained above so as, forming a layer of inert substance thereon with a composition in mg/tablet amounting to mg:

| | | |
|---|---|---|
| Mannitol | 307.40 | (96.06%) |
| Povidone | 8.60 | (2.69%) |
| Magnesium stearate | 4.00 | (1.25%) |

The tablets thus obtained are coated in a drum mixer with PVA-PEG copolymer in an 18% aqueous suspension until they increase in weight by 11 mg per tablet.

The composition of the PVA-PEG copolymer comprises (mg/tablet and percentages):

| | | |
|---|---|---|
| PVA | 5.33 | (48.48%) |
| Titanium dioxide | 1.54 | (14.00%) |
| PEG | 1.68 | (15.26%) |
| Kaolin | 1.76 | (16.00%) |
| PVP | 0.45 | (4.08%) |
| Sodium lauryl sulphate | 0.22 | (2.00%) |
| Anhydrous colloidal silica | 0.02 | (0.18%) |

Finally, the resulting tablets are further coated with a gastroresistant film in a 24.2% aqueous suspension until they increase in weight by 11 mg per tablet. This coating has the following composition (mg/tablet and percentages):

| | | |
|---|---|---|
| Eudragit L100-55 | 6.82 | (62%) |
| Talc | 3.41 | (31%) |
| Triethyl citrate | 0.68 | (6.18%) |
| Sodium hydroxide | 0.09 | (0.82%) |

### Example 2

First granules containing rabeprazole sodium, povidone and polysorbate 80 are prepared using the fluid bed (as explained in example 1).

These granules are mixed in a manual drum mixer with magnesium stearate as a lubricant and small tablets, 3 mm in diameter, are produced with the following unit composition, in mg:

| | | |
|---|---|---|
| Rabeprazole sodium | 10.00 | (86.96%) |
| Povidone | 0.81 | (7.04%) |
| Polysorbate 80 | 0.19 | (1.65%) |
| Magnesium stearate | 0.50 | (4.35%) |

Second inert granules are prepared separately in a fluid bed containing mannitol, which is sprayed with a solution of povidone-ethanol (1:9). After spraying with all of the previously-described solution, the granules are dried inside the same fluid bed.

After drying, the resulting granules are forced through a vibrating sieve fitted with a mesh with holes 1000 μm in diameter and mixed in a drum mixer with magnesium stearate as a lubricant.

Using a suitable compression machine, the previously-prepared inner tablets are covered with the second granules, forming a layer of inert substance thereon with a composition in mg/tablet and percentages amounting to:

| | | |
|---|---|---|
| Mannitol | 307.40 | (96.06%) |
| Povidone | 8.60 | (2.69%) |
| Magnesium stearate | 4.00 | (1.25%) |

The resulting tablets are coated only with a gastroresistant film in a 24.2% aqueous suspension until they increase in weight by 12 mg per tablet. This coating has the following composition (mg/tablet and percentages):

| | | |
|---|---|---|
| Eudragit L100-55 | 7.44 | (62%) |
| Talc | 3.72 | (31%) |
| Triethyl citrate | 0.74 | (6.18%) |
| Sodium hydroxide | 0.10 | (0.82%) |

### Example 3

First granules containing rabeprazole sodium, povidone and polysorbate 80 are prepared using a wet granulation technique, spraying over these powders a solution of povidone-polysorbate 80-ethanol (1;0.23;9), then dried in a cupboard under forced air circulation. After drying, the resulting granules are forced through a vibrating sieve fitted with a mesh with holes 1000 μm in diameter.

The product obtained is mixed in a manual drum mixer with magnesium stearate as a lubricant and then used to prepare small tablets, 3 mm in diameter, with a unit composition similar to the one described in examples 1 and 2.

The resulting tablets are then coated in a drum mixer with PVA-PEG copolymer (as explained in example 1 above) in an 18% aqueous suspension until they increase in weight by 30 mg per tablet.

Again using a wet granulation process, second inert granules containing mannitol are prepared separately, spraying them with a solution of povidone-ethanol (1:9).

After drying in a cupboard under forced air circulation, the resulting granules are forced through a vibrating sieve fitted with a mesh with holes 1000 μm in diameter and mixed in a manual drum mixer with magnesium stearate as a lubricant.

Using a suitable compression machine, the previously-prepared inner tablets are covered with the second granules, forming a layer of inert substance thereon with a composition in mg/tablet and percentages amounting to:

| | | |
|---|---|---|
| Mannitol | 307.40 | (96.06%) |
| Povidone | 8.60 | (2.69%) |
| Magnesium stearate | 4.00 | (1.25%) |

The resulting tablets are coated only with a gastroresistant film in a 24.2% aqueous suspension until they increase in weight by 12 mg per tablet. This coating has the following composition (mg/tablet and percentages):

| | | |
|---|---|---|
| Eudragit L100-55 | 7.44 | (62%) |
| Talc | 3.72 | (31%) |
| Triethyl citrate | 0.74 | (6.18%) |
| Sodium hydroxide | 0.10 | (0.82%) |

### Example 4

Using the fluid bed, first granules containing rabeprazole sodium, povidone and polysorbate 80 are prepared as described in example 1.

The granules are mixed in a drum mixer with magnesium stearate as a lubricant and used to produce small tablets, 3 mm in diameter, with a unit composition, in mg, of:

| | | |
|---|---|---|
| Rabeprazole sodium | 20.00 | (86.96%) |
| Povidone | 1.62 | (7.04%) |
| Polysorbate 80 | 0.38 | (1.65%) |
| Magnesium stearate | 1.00 | (4.35%) |

Second inert granules are prepared separately in a fluid bed containing mannitol, which is sprayed with a solution of povidone-ethanol (1:9). After spraying with all of the previously-described solution, the granules are dried inside the same fluid bed.

After drying, the resulting granules are forced through a vibrating sieve fitted with a mesh with holes 1000 μm in diameter and mixed in a manual drum mixer with magnesium stearate as a lubricant.

Using a suitable compression machine, the inner tablets containing the first granules are covered with the second granules combined, forming a layer of inert substance thereon with a composition in mg/tablet and percentages amounting to:

| | | |
|---|---|---|
| Mannitol | 384.00 | (96%) |
| Povidone | 11.00 | (2.75%) |
| Magnesium stearate | 5.00 | (1.25%) |

The tablets obtained are coated in a drum mixer with PVA-PEG copolymer (as described in example 1) in an 18% aqueous suspension until they increase in weight by 12 mg per tablet.

The resulting tablets are coated with a gastroresistant film in a 24.2% aqueous suspension until they increase in weight by 10 mg per tablet. This coating has the following unit composition (mg/tablet and percentages):

| | | |
|---|---|---|
| Eudragit L100-55 | 6.2 | (62%) |
| Talc | 3.1 | (31%) |
| Triethyl citrate | 0.62 | (6.2%) |
| Sodium hydroxide | 0.08 | (0.8%) |

### Example 5

Using a wet granulation technique, first granules containing rabeprazole sodium, povidone and polysorbate 80 are prepared, spraying the powders with a solution of povidone-polysorbate 80-ethanol (1;0.23;9), then drying the product in a cupboard under forced air circulation. After drying, the resulting granules are forced through a vibrating sieve fitted with a mesh with holes 1000 μm in diameter. The granules are mixed in a manual drum mixer with magnesium stearate as a lubricant; this mixture is then used to produce small tablets, 3 mm in diameter, with a unit composition, in mg, of:

| | | |
|---|---|---|
| Rabeprazole sodium | 20.00 | (86.96%) |
| Povidone | 1.62 | (7.04%) |
| Polysorbate 80 | 0.38 | (1.65%) |
| Magnesium stearate | 1.00 | (4.35%) |

Second inert granules are prepared separately in a fluid bed containing mannitol, which is sprayed with a solution of povidone-ethanol (1:9). After spraying with all of the previously-described solution, the granules are dried inside the same fluid bed.

After drying, the resulting granules are forced through a vibrating sieve fitted with a mesh with holes 1000 μm in diameter and mixed in a manual drum mixer with magnesium stearate as a lubricant.

Using a suitable compression machine, the tablets containing the first granules are covered with the second granules combined, forming a layer of inert substance thereon with a composition in mg/tablet and percentages amounting to:

| | | |
|---|---|---|
| Mannitol | 384.00 | (96%) |
| Povidone | 11.00 | (2.75%) |
| Magnesium stearate | 5.00 | (1.25%) |

The resulting tablets are coated only with a gastroresistant film in a 24.2% aqueous suspension until they increase in weight by 13 mg per tablet. This coating has the following unit composition (mg/tablet and percentages):

| | | |
|---|---|---|
| Eudragit L100-55 | 8.06 | (62%) |
| Talc | 4.03 | (31%) |
| Triethyl citrate | 0.80 | (6.15%) |
| Sodium hydroxide | 0.11 | (0.85%) |

### Example 6

Pantoprazole sodium sesquihydrate, mannitol and crospovidone are placed in a fluid bed with a top-spray insert and sprayed first with a solution of polysorbate 80-ethanol (1:10) and then only with purified water (in a quantity equating to half the total weight of the powders).

After spraying with all the previously-described solutions, the granules are dried in the same fluid bed.

After drying, the resulting granules are forced through a vibrating sieve fitted with a mesh with holes 1000 μm in diameter and mixed in a manual drum mixer with magnesium stearate as a lubricant.

The above-described mix of granules is used to produce small tablets, 3 mm in diameter, with a unit composition (in mg/tablet and percentages) of:

| | | |
|---|---|---|
| Pantoprazole sodium sesquihydrate | 20.00 | (75.47%) |
| Crospovidone | 3.00 | (11.32%) |
| Mannitol | 2.00 | (7.55%) |
| Polysorbate 80 | 1.00 | (3.77%) |
| Magnesium stearate | 0.50 | (1.89%) |

Second inert granules are prepared separately in a fluid bed containing mannitol and crospovidone, sprayed with a solution of povidone-ethanol (1:9). After spraying with all of the previously-described solution, the granules are dried inside the same fluid bed.

After drying, the resulting granules are forced through a vibrating sieve fitted with a mesh with holes 1000 μm in diameter and mixed in a manual drum mixer with magnesium stearate as a lubricant.

Using a suitable compression machine, the inner tablets containing the first granules are covered with the second mix of granules so as, forming a layer of inert substance thereon with a composition (in mg/tablet and percentages) amounting to:

| | | |
|---|---|---|
| Mannitol | 379.00 | (91.33%) |
| Crospovidone | 20.00 | (4.82%) |
| Povidone | 11.00 | (2.65%) |
| Magnesium stearate | 5.00 | (1.20%) |

The tablets thus obtained are coated in a drum mixer with PVA-PEG copolymer (as described in example 1) in an 18% aqueous suspension until they increase in weight by 17.3 mg per tablet.

The resulting tablets are then further coated with a gastroresistant film in a 24.2% aqueous suspension until they increase in weight by 20 mg per tablet. This coating has the following composition (in mg/tablet and percentages):

| | | |
|---|---|---|
| Eudragit L100-55 | 12.40 | (62%) |
| Talc | 6.20 | (31%) |
| Triethyl citrate | 1.24 | (6.2%) |
| Sodium hydroxide | 0.16 | (0.8%) |

### Example 7

Using the fluid bed, granules containing pantoprazole sodium sesquihydrate, mannitol, crospovidone and polysorbate 80 are prepared as described in example 6.

The granules are mixed in a manual drum mixer with magnesium stearate as a lubricant and then used to produce small tablets, 6 mm diameter, with a unit composition (in mg/tablet and percentages) of:

| | | |
|---|---|---|
| Pantoprazole sodium sesquihydrate | 40.00 | (75.47%) |
| Crospovidone | 6.00 | (11.32%) |
| Mannitol | 4.00 | (7.55%) |
| Polysorbate 80 | 2.00 | (3.77%) |
| Magnesium stearate | 1.00 | (1.89%) |

Second inert granules containing mannitol and crospovidone are prepared separately in a fluid bed, where they are sprayed with a solution of povidone-ethanol (1:9). After spraying with all of the previously-described solution, the granules are dried inside the same fluid bed.

After drying, the resulting granules are forced through a vibrating sieve fitted with a mesh with holes 1000 μm in diameter and mixed in a manual drum mixer with magnesium stearate as a lubricant.

Using a suitable compression machine, the inner tablets containing the first granules are covered with the second granules combined so as, forming a layer of inert substance thereon with a composition (in mg/tablet and percentages) amounting to:

| | | |
|---|---|---|
| Mannitol | 347.00 | (91.32%) |
| Crospovidone | 18.20 | (4.8%) |
| Povidone | 10.20 | (2.68%) |
| Magnesium stearate | 4.60 | (1.20%) |

The resulting tablets are coated only with a gastroresistant film in a 24.2% aqueous suspension until they increase in weight by 20 mg per tablet. This coating has the following composition (in mg/tablet and percentages):

| | | |
|---|---|---|
| Eudragit L100-55 | 12.40 | (62%) |
| Talc | 6.20 | (31%) |
| Triethyl citrate | 1.24 | (6.2%) |
| Sodium hydroxide | 0.16 | (0.8%) |

### Example 8

Esomeprazole magnesium dihydrate, croscarmellose sodium and calcium silicate are placed in a fluid bed with a top-spray insert and are sprayed first with a solution of povidone (1:10) in isopropanol, and then with isopropanol alone.

After spraying with the previously-described solutions, the granules are dried in the same fluid bed.

After drying, the resulting granules are forced through a vibrating sieve fitted with a mesh with holes 1000 μm in diameter.

The product is mixed in a manual drum mixer with magnesium stearate, anhydrous colloidal silica, Ludiflash* and croscarmellose sodium.

This mix of granules is used to produce small tablets, 5 mm in diameter, with a unit composition (in mg/tablet and percentage) of:

| | | |
|---|---|---|
| Esomeprazole magnesium dihydrate | 20.00 mg | (41.67%) |
| Croscarmellose sodium | 1.60 mg | (3.33%) |
| Calcium silicate | 6.30 mg | (13.13%) |
| Povidone | 8.10 mg | (16.88%) |
| Magnesium stearate | 0.50 mg | (1.04%) |
| Anhydrous colloidal silica | 0.50 mg | (1.04%) |
| Ludiflash | 8.00 mg | (16.67%) |
| Croscarmellose sodium | 3.00 mg | (6.24%) |

(where Ludiflash indicates a mixture of: mannitol, crospovidone, polyvinyl acetate and povidone).

Microcrystalline cellulose, isomalt and magnesium stearate (as a lubricant) are separately combined in a mixer for powders and these powders are mixed together to obtain a homogeneous, flowing mixture.

Using a suitable compression machine, the inner tablets made with the first granules are covered with the second mix of granules forming a layer of inert substance thereon and obtaining an elongated tablet 14×8 in size.

The inert layer has the following composition (in mg/tablet and percentages):

| | | |
|---|---|---|
| Microcrystalline cellulose | 497.00 mg | (88%) |
| Isomalt | 5.50 mg | (11%) |
| Magnesium stearate | 1.50 mg | (1%) |

The resulting tablets are coated with a gastroresistant film in a 24.2% aqueous suspension until they increase in weight by 30 mg per tablet. This coating has the following composition (mg/tablet and percentages):

| | | |
|---|---|---|
| Eudragit L100-55 | 18.60 | (62%) |
| Talc | 9.30 | (31%) |
| Triethyl citrate | 1.86 | (6.2%) |
| Sodium hydroxide | 0.24 | (0.8%) |

### Example 9

Esomeprazole magnesium dihydrate, croscarmellose sodium and calcium silicate are placed in a fluid bed fitted with a top-spray insert and sprayed first with a solution of povidone (1:10) in isopropanol and then with isopropanol alone, as in example 8.

After spraying with all the previously described solutions, the granules are dried in the same fluid bed.

After drying, the resulting granules are forced through a vibrating sieve fitted with a mesh with holes 1000 μm in diameter.

The mix of granules is used to produce small tablets, 6 mm in diameter, with a unit composition (in mg/tablet and percentages) of:

| | | |
|---|---|---|
| Esomeprazole magnesium dihydrate | 40.00 | (41.67%) |
| Croscarmellose sodium | 13.20 | (3.33%) |
| Calcium silicate | 12.60 | (13.13%) |
| Povidone | 16.20 | (16.88%) |
| Magnesium stearate | 1.00 | (1.04%) |
| Anhydrous colloidal silica | 1.00 | (1.04%) |
| Ludiflash | 16.00 | (16.67%) |
| Croscarmellose sodium | 6.00 | (6.24%) |

Second inert granules containing mannitol and crospovidone are prepared separately in a fluid bed and sprayed with a solution of povidone-ethanol (1:9). After spraying with all of the previously-described solution, the granules are dried inside the same fluid bed.

After drying, the resulting granules are forced through a vibrating sieve fitted with a mesh with holes 1000 μm in diameter and mixed in a manual drum mixer with magnesium stearate as a lubricant.

Using a suitable compression machine, the inner tablets made with the first granules are covered with the second mix of granules, forming a layer of inert substance thereon and obtaining an elongated tablet 16×9 in size.

The inert layer has the following composition (in mg/tablet and percentages):

| | | |
|---|---|---|
| Mannitol | 642.00 | (91.19%) |
| Crospovidone | 34.00 | (4.83%) |
| Povidone | 18.00 | (2.57%) |
| Magnesium stearate | 10.00 | (1.41%) |

The resulting tablets are coated with a gastroresistant film in a 24.2% aqueous suspension until they increase in weight by 60 mg per tablet. This coating has the following composition (mg/tablet and percentages):

| | | |
|---|---|---|
| Eudragit L100-55 | 37.20 | (62%) |
| Talc | 18.60 | (31%) |
| Triethyl citrate | 3.72 | (6.2%) |
| Sodium hydroxide | 0.48 | (0.8%) |

1. A pharmaceutical formulation for the oral administration of proton pump inhibitors comprising:
an inner tablet containing the proton pump inhibitor;
an outer protective tablet surrounding the previous inner tablet;
a gastroresistant, or protective and gastroresistant coating;
and possibly also a protective film in between the above-mentioned inner tablet and outer protective tablet

2. The formulation according to claim 1 wherein the proton pump inhibitor is chosen in the group consisting of: omeprazole, pantoprazole sodium sesquihydrate, lansoprazole, rabeprazole sodium, esomeprazole magnesium dihydrate and trihydrate.

3. Formulations according to claim 2, wherein said inner tablet containing the active ingredient is a conventional tablet comprising the usual surfactants, binders and disintegrants normally used for such purposes, in addition to the active ingredient.

4. The formulation according to claim 3, wherein said surfactants, binders and disintegrants are chosen in the group consisting of: mannitol, povidone and its derivatives, calcium silicate, microcrystalline cellulose, sorbitol, lactose, starch and its derivatives.

5. The formulation according to claim 4, wherein the inner tablet has the following composition:

| | | | |
|---|---|---|---|
| active ingredient | 50-80% | preferably | 60-80% |
| surfactants | 1-15% | preferably | 3-10% |
| binders | 3-25% | preferably | 5-15% |
| disintegrants | 1-10% | preferably | 4-6% |
| diluents | 15-30% | preferably | 8-20% |

6. The formulation according to claim 1, wherein the protective tablet consists of granules comprising: polyalcohols, pharmaceutically-allowable soluble or insoluble polymers, and lubricants.

7. The formulation according to claim 6, wherein said polyalcohols are chosen in the group consisting of: mannitol, polyisosorbate, isomalt, xylitol, erythritol.

8. Formulations according to claim 7, wherein said pharmaceutically-allowable polymers are chosen from among the following: povidone, crospovidone, polyethylene glycol, polyvinyl alcohol, cellulose derivatives and modifications thereof.

9. The formulation according to claim 6, wherein the protective tablet has the following composition:

| | | | |
|---|---|---|---|
| binders | 5-10% | preferably | 6-10% |
| disintegrants | 14-30% | preferably | 18-25% |
| diluents | 50-80% | preferably | 60-75% |
| lubricants | 1-5% | preferably | 1-3% |

10. The formulation according to claim 1, wherein said lubricants are chosen in the group consisting of: magnesium stearate, talc, colloidal silica, and so on.

11. The formulations according to claim 9, wherein the protective layer applied to the outer tablet consists of pH-dependent or pH-independent films.

12. A process for the preparation of a formulations according to claim 1, wherein:
granules containing the active ingredient are prepared using a fluid bed or a conventional granulator, then the granules thus obtained are mixed with the necessary lubricant excipients, and the mixture is compressed into the form of a tablet or, alternatively, the mixture of active ingredient, excipients and lubricant is obtained by direct compression;
inert granules to form the protective tablet are prepared and the resulting granules, possibly mixed with other lubricant excipients, are compressed into the form of a tablet around the previously-obtained tablets;
the resulting tablet is coated with a layer of gastroresistant film, possibly preceded by a layer of a coating for protecting it against humidity.
